(19) United States
(12) Patent Application Publication
(10) Pub. No.: US 2008/0286801 A1
(43) Pub. Date: Nov. 20, 2008

(54) Method for the analysis of differential expression in colorectal cancer

(76) Inventors: Carlos Buesa Arjol, Barcelona (ES); Simo Schwartz Navarro, Barcelona (ES); Diego Arango Del Corro, Barcelona (ES)

Correspondence Address: TRASK BRITT, P.O. BOX 2550, SALT LAKE CITY, UT, 84110, US

(21) Appl. No.: 12/214,522

(22) Filed: Jun. 18, 2008

Related U.S. Application Data
(63) Continuation-in-part of application No. PCT/ES2006/000704, filed on Dec. 19, 2006.

(30) Foreign Application Priority Data
Dec. 21, 2005 (ES) P 200503203

(51) Int. Cl. C12Q 1/68; G01N 33/574

(52) U.S. Cl. 435/6; 435/7.23

(57) ABSTRACT

A method for the analysis of differential expression in colorectal cancer based on the variation in the expression levels of genes encoding for proteins forming part of the condensin complex or associated proteins that occurs in patients with the disease and that can be used as markers for the diagnosis of the cancers, as well as for the prevention and treatment thereof.

### CROSS-REFERENCE TO RELATED APPLICATIONS

This application is a continuation-in-part of and claims priority to PCT Application No. PCT/ES2006/000704 (publication number WO2007/074193) filed Dec. 19, 2006, which claims priority to Spanish Application No. P 200503203 filed Dec. 21, 2005, each of which is hereby incorporated herein by reference.

### FIELD OF THE INVENTION

The present invention relates to a method for the analysis of differential expression, based on the over-expression of proteins of the condensin complex and associated proteins in colorectal cancer patients, which can be used as a criterion for the diagnosis of these cancers, as well as in the prevention and treatment thereof.

### BACKGROUND OF THE INVENTION

In absolute terms, cancer is the second most common cause of death in Spain. Among the many types of cancer, colorectal cancer stands out, according to 1999 data, as having been responsible for 11% of cancer deaths in men and 15% in women. In Spain, the estimated annual number of new cases of both sexes stands at around 18,000, as against 11,300 deaths. Owing to frequent errors in classifying tumors of the rectosigmoid portion, colon and rectal tumors are generally treated as one for analysis purposes. Mortality from colorectal cancer is very high, this being the second most common form of cancer in both men and women, with the trend rising with age (2.2% annually in men and 0.7% in women). Nowadays mortality is higher in men, though in the 1960s it was higher in women.

With these tumors, mortality data do not reflect the true incidence of the disease, given that survival has improved in recent years, primarily in young people. The trend toward stabilization of mortality may reflect the therapeutic improvements achieved with early diagnosis, given that the tumors in question are fairly accessible to sigmoidoscopic examination and the universal use of full colonoscopies for screening identified risk groups.

The cumulative lifetime risk of contracting the disease is 5% to 6%, depending on lifestyles and hereditary factors. The most common colorectal cancer is the sporadic type (90%), and some cases having elements of inherited predisposition: familial adenomatous polyposis (0.01%) and hereditary nonpolyposis colorectal cancer (5% to 10%). The latter are caused by familial syndromes defined by just a few genes. However, the genes responsible for sporadic cancers have yet to be identified. It is believed that a colorectal tumor may develop as a consequence of a series of molecular events that start off with one or more mutations or epigenetic events and continue with progression phenomena in which both genetic and environmental factors may be involved.

Generally speaking, with colorectal cancer, the symptoms usually only become apparent in the advanced stages of growth in the intestinal wall, which makes it necessary to identify new genes and/or mutations responsible for and/or indicative of this type of cancer and to stimulate the consequent development of analytical methods that will allow selective and rapid molecular diagnosis of the disease in order that they can eventually form part of routine clinical practice.

This would also lead to savings in healthcare costs and reduced waiting times, to the adoption of new prognostic criteria and treatment approaches in positive cases, and to the design of selective therapies associated with these genes.

The protein encoded by the gene SMC2L1 (human ortholog of SMC2 of S. cerevisiae, also called hCAP-E) is a crucial protein in chromosome condensation and compaction complexes that could perform functions related to the regulation of gene transcription (Hagstrom and Meyer, 2003; Hirano, 2002; Legagneux et al., 2004).

This protein belongs to a family of so-called SMC proteins (standing for “structural maintenance of chromosomes”), which comprises six proteins numbered from 1 to 6 (SMC1, SMC2, SMC3, SMC4, SMC5, and SMC6). These proteins form dimers with other SMC proteins and active complexes with other so-called non-SMC proteins (divided into two families, HEAT and kleisin). Overall, they form three primary complexes: condensin (in humans there are two differentiated complexes, condensin I and condensin II), cohesin and the hSMC5-hSMC6 complex, associated with genomic repair (Hagstrom and Meyer, 2003; Hirano, 2002). The proteins and the complexes they form in different species are described in Tables 1 and 2.

TABLE 1
Structural maintenance of chromosomes

| | | | | | |
|---|---|---|---|---|---|
| Saccharomyces | Schizosaccharomyces | Caenorhabditis | Drosophila | Xenopus | Homo |
| cerevisiae | pombe | elegans | melanogaster | laevis | sapiens |

Cohesin

| | | | | | | |
|---|---|---|---|---|---|---|
| SMC1 | Smc1 | Psm1 | HIM-1 | SMC1 | SMC1 | SMC1 |
| SMC3 | Smc3 | Psm3 | SMC-3 | SMC3 | SMC3 | SMC3 |
| SCC1 | Scc1/Mcd1 | Rad21 | SCC-1/COH-2 | Rad21 | RAD21 | RAD21 |
| SCC3 | Scc3 | Psc3 | SCC-3 | SA | SA1, SA2 | SA1, SA2 |

Condensin

| | | | | | | |
|---|---|---|---|---|---|---|
| SMC2 | Smc2 | Cut14 | MIX-1 | SMC2 | CAP-E | CAP-E |
| SMC4 | Smc4 | Cut3 | SMC-4 | SMC4/gluon | CAP-C | CAP-C |
| CAP-D2 | Ycs4 | Cnd1 | HCP-6 | CG1911 | CAP-D2 | CAP-D2 |
| CAP-G | Ycs5/Ycg1 | Cnd3 | — | CG17054 | CAP-G | CAP-G |
| CAP-H | Brn1 | Cnd2 | DPY-26 | Barren | CAP-H | CAP-H |

DNA repair

| | | | | | | |
|---|---|---|---|---|---|---|
| SMC5 | Smc5 | Spr18 | C27A2.1 | CG3248 | SMC5 | SMC5 |
| SMC6 | Smc6/Rhc18 | Rad18 | C23H4.6 | CG5524 | SMC6 | SMC6 |

(Adapted from Hagstrom and Meyer, 2003)

TABLE 2
Condensin complexes

| | | | | | Kleisin |
|---|---|---|---|---|---|
| | SMC subunits | | HEAT-repeat subunits | | subunits |
| Condensin S. cerevisiae | Smc2 | Smc4 | Ycs4p | Ycs5p/Ycg1p | Brn1 |
| Condensin S. pombe | Cut14 | Cut3 | Cnd1 | Cnd3 | Cnd2 |
| Condensin I D. melanogaster | SMC2 | SMC4/Gluon | CG1911 | CG17054 | Barren |
| Condensin I C. elegans | MIX-1 | DPY-27 | DPY-28 | ? | DPY-26 |
| Condensin I C. elegans | MIX-1 | SMC-4 | HCP-6 | ? | KLE-2/C2 |
| | | | | | 9E4.2 |
| Condensin I X. laevis | XCAP-E | XCAP-C | XCAP-D2 | XCAP-G | XCAP-H |
| Condensin II X. laevis | XCAP-E | XCAP-C | XCAP-D3 | XCAP-G2 | XCAP-H2 |
| Condensin I H. sapiens | hCAP-E | hCAP-C | hCAP-D2 | hCAP-G | hCAP-H |
| Condensin II H. sapiens | hCAP-E | hCAP-C | hCAP-D3 | hCAP-G2 | hCAP-H2 |

SMC2L1 forms part of the condensin I and II complexes. Both complexes perform functions relating to chromatin compaction (Hirano et al., 1994; Ono et al., 2004; Ono et al., 2003):
Condensin I: formed by the dimer SMC2-SMC4 (hCAP-E-hCAP-C), the HEAT subunits hCAP-D2/CNAP1 and hCAP-G, and the kleisin subunit hCAP-H. The dimer hCAP-E-hCAP-C is located in the cytoplasm of interphase cells (except during mitosis). Once mitosis has occurred, the non-SMC subunits undergo phosphorylation, interact with the hCAP-E-hCAP-C dimer, form the complex, and are transferred to the nucleus, where they interact with the DNA and compact it (Hagstrom and Meyer, 2003).
Condensin II: formed by the dimer SMC2-SMC4 (hCAP-E-hCAP-C), HEAT subunits hCAP-D3 and hCAP-G, and kleisin subunit hCAP-H2. It seems that the complex is located in the nucleus of interphase cells (FIG. 1A) and could be associated with the regulation of gene transcription, helping to compact the promoter region of certain genes. This would place condensin in the euchromatin regions associated with transcriptionally active regions (regions of transcriptional regulation, i.e., of transcription activation or inhibition), as the regions equivalent to the internal nuclear region (FIGS. 1A and 1B) and the chromosome R-bands, with other regulatory factors such as acetylated histones (for example, acetylated histone H3 at lysine K3) (FIGS. 2 and 3).

There is no evidence in the literature relating these proteins with this type of cancer. However, the possible link between condensin and transcriptional silencing and proper chromosome compaction suggests that it could be altered in cancer. Most cancers present general genomic hypomethylation accompanied by general hyperacetylation, but it has been shown that certain gene promoter regions rich in CpG islands are hypermethylated and the genes are silenced (are not transcribed). In the present invention, it is described how condensin is involved in these silencing processes and how the proteins forming these complexes and associated proteins are over-expressed. Similarly, given that it is believed that the majority of epigenetic alterations occur in the very early stages of neoplastic processes, this over-expression may also be characteristic of early processes such as adenomas.

### DISCLOSURE OF THE INVENTION

The invention relates to the discovery that certain genes (and the proteins they encode) have altered expression levels in colorectal cancer. In particular, the studies disclosed herein demonstrate that members of the condensin complexes (and associated proteins) like hCAP-E, hCap-C, hCap-D2, hCap-D3, hCap-G, hCap-G2, hCap-H, hCap-H2, and KIF4A, have altered expression levels in cancer, e.g., adenomas and colorectal cancer. The invention, therefore, provides a method for detecting biomarkers that correspond to the expression of the level of genes that encode proteins that form a part of, or are associated with, the condensin complex for the diagnosis cancer. For example, biomarkers that can correspond to the level of expression of one or more genes of the condensin complex include the proteins and/or genes encoding the proteins of the condensin I complex, the proteins and/or genes encoding the proteins of the condensin II complex, and/or proteins and the genes that encode proteins associated with these complexes. Alterations in the expression level of one or more genes that encode proteins that are part of the condensin complexes (or that are associated with the complex) as compared to normal tissue can indicate a cancerous or precancerous condition. In some aspects of the invention, the levels of mRNA are determined, while in other aspects, the level of proteins are determined. mRNA levels can be determined by any method available to the skilled artisan, e.g., by quantitative PCR or microarray-based detection. Protein levels can be determined by any method available to the skilled artisan, e.g., by antibody-based detection (including, but not limited to, ELISA and immunohistochemistry). The invention also provides methods for screening for compounds that alter the expression level and/or function of the condensin complex biomarkers.

In one embodiment, the invention provides a method for the diagnosis colorectal cancer. According to one aspect of this embodiment, the method involves (1) obtaining a sample from an individual and (2) detecting the level of one or more biomarkers corresponding to genes encoding one or more proteins that are a part of, or are associated with, the condensin complexes. According to one aspect of this embodiment, the one or more biomarkers are chosen from those corresponding to genes encoding protein (or the proteins themselves) that form the condensin I complex, those that are associated with the condensin I complex, those that form the condensin II complex, and those that are associated with the condensin II complex. According to one aspect of this embodiment, the one or more biomarkers that are detected correspond to and are selected from hCAP-E, hCap-C, hCap-D2, hCap-D3, hCap-G, hCap-G2, hCap-H, hCap-H2, and KIF4A. In one aspect of this embodiment, the level of the biomarker in the tissue being investigated (e.g., cancerous tissue or tissue suspected of being cancerous) is compared to the level of the biomarker in normal tissue. If the level of the biomarker is increased compared to normal tissue, then the tissue is likely to be cancerous or premalignant. In an alternative format, a reference value for the level of biomarker can be established and the value of the level of the biomarker in the tissue being analyzed can be compared to this reference value.

In another embodiment, the invention provides a method for the diagnosis of colorectal cancer. According to one aspect of this embodiment, the method involves obtaining a sample from an individual and detecting the level of one or more nucleic acids that encode protein(s) that are a part of, or are associated with, the condensin complexes. According to one aspect of this embodiment, the one or more nucleic acids that encode protein(s) that are a part of, or are associated with the condensin complexes correspond to and are selected from hCAP-E, hCap-C, hCap-D2, hCap-D3, hCap-G, hCap-G2, hCap-H, hCap-H2, and KIF4A. In one aspect of this embodiment, the level of the nucleic acid(s) in the tissue being investigated (e.g., cancerous tissue or tissue suspected of being cancerous) is compared to the level of the nucleic acid in normal tissue. If the level of the nucleic acid in the sample tissue is increased compared to normal tissue, then the sample tissue is likely to be cancerous or premalignant. In an alternative format, a reference value for the level of the nucleic acid can be established and the value of the nucleic acid in the tissue being analyzed can be compared to this reference value.

In another embodiment, the invention provides a method for the diagnosis of colorectal cancer. According to one aspect of this embodiment, the method involves obtaining a sample from an individual and detecting the level of one or more proteins that are a part of or are associated with the condensin complex. According to one aspect of this embodiment, the one or more proteins that are detected are selected from hCAP-E, hCap-C, hCap-D2, hCap-D3, hCap-G, hCap-G2, hCap-H, hCap-H2, and KIF4A. In one aspect of this embodiment, the level of the protein(s) in the tissue being investigated (e.g., cancerous tissue or tissue suspected of being cancerous) is compared to the level of the protein in normal tissue. If the level of the protein in the sample tissue is increased compared to normal tissue, then the sample tissue is likely to be cancerous or premalignant. In an alternative format, a reference value for the level of the protein expression can be established and the value of expression in the tissue being analyzed can be compared to this reference value.

In another embodiment, the invention provides a method for identifying compounds that may be useful for treating cancer. According to one aspect of this embodiment, the method involves treating a sample with a test compound and detecting the level of one or more proteins (or mRNA(s) encoding the protein) that are a part of or are associated with the condesin complex. If the test compound reduces the level of the protein (or mRNA encoding the protein), then it is identified that it can be used to treat colorectal cancer.

According to one aspect of this embodiment, the one or more proteins chosen that are part of or are associated with the condensin complex are selected from hCAP-E, hCap-C, hCap-D2, hCap-D3, hCap-G, hCap-G2, hCap-H, hCap-H2, and KIF4A. In one aspect of this embodiment, the level of the protein(s) in the tissue being investigated (e.g., cancerous or suspected of being cancerous) is compared to the level of the protein in normal tissue. If the level of the protein is increased compared to normal tissue, then the tissue is likely to be cancerous or premalignant.

In one embodiment of the invention, the invention provides a method for detecting pluripotent stem cells or cells that have pluripotent characteristics. According to this embodiment, a sample is obtained from an individual and the level of an mRNA encoding one or more proteins that are a part of or are associated with the condesin complex is determined. According to one aspect of this embodiment, the one or more proteins that are part of or are associated with the condensin complex are selected from hCAP-E, hCap-C, hCap-D2, hCap-D3, hCap-G, hCap-G2, hCap-H, hCap-H2, and KIF4A. If the level of the mRNA is increased compared to a control or normal tissue, then the cell is more likely to be pluripotent or have a more pluripotent phenotype. An alternative method involves determining the level of protein.

It is the object of the present invention to provide a method for the analysis of differential expression in colorectal cancer, comprising the determination, in a biological sample isolated from a patient, of a variation in the expression levels of one or more protein-encoding genes forming part of the condensin complex or other proteins interacting with the complex, wherein the variation in gene expression levels is used to diagnose for the presence of colorectal cancer or of a premalignant condition thereof.

In one embodiment, the gene or genes (or the proteins they encode) are selected from those that form part of the condensin complex or proteins associated with the condensin complex. In a more specific embodiment, the gene or genes (or the proteins they encode) to be analyzed are selected from among the group consisting of hCap-E, hCap-C, hCap-D2, hCap-D3, hCap-G, hCap-G2, hCap-H, hCap-H2, and KIF4A and the variation in the expression levels of the gene or genes that is indicative of cancer or a premalignant state is an increase in expression levels.

In one embodiment, the sample to be analyzed is a biological sample. In a more specific embodiment, the biological sample is chosen from a tumor, a tumor block, tumor section, tumor tissue, cancer cells, cells suspected of being cancerous, cells, a biopsy, a stool sample, a blood sample, and a body fluid sample. In a specific embodiment, the sample can comprise nucleic acids, DNA, RNA, and/or protein, and can be isolated from a biological sample (e.g., cells obtained by biopsy) or any other method of extraction.

In an embodiment of the invention, the step of determining or analyzing the one or more genes comprises nucleic acid amplification. In a specific aspect of this embodiment, the amplification is carried out by PCR amplification, SDA amplification, or any other method of nucleic acid amplification. In a specific aspect of this embodiment, the one or more genes (e.g., mRNA) are amplified by PCR. In another specific aspect of this embodiment, the level of one or more genes is determined using quantitative PCR. In a specific aspect of this embodiment the levels of expression of one or more genes chosen from hCap-E, hCap-C, hCap-D2, hCap-D3, hCap-G, hCap-G2, hCap-H, hCap-H2, and KIF4A, are evaluated.

In another embodiment of the invention, the step of determining or analyzing the one or more genes comprises using a DNA biochip (e.g., expression microarray). In some specific aspects, the DNA biochip (e.g., microarray) is made with oligonucleotides deposited by any mechanism or by means of DNA biochips made with oligonucleotides synthesized in situ by photolithography or any other mechanism.

In another embodiment of the invention, the step of determining or analyzing the one or more genes comprises in situ hybridization using specific probes labeled using any labeling method.

In another embodiment of the invention, the step of determining or analyzing the one or more genes comprises gel electrophoresis. Optionally, the determination may be carried out by transfer to a membrane and hybridization with a specific probe.

In another embodiment of the invention, the determination is carried out by NMR or any other diagnostic imaging technique.

In another embodiment of the invention, the determination is carried out by NMR or any other diagnostic imaging technique and the use of paramagnetic nanoparticles or any other type of detectable nanoparticles functionalized with antibodies or any other means.

In yet another embodiment, the step of analyzing or determining the level of one or more genes comprises determining the level of the protein encoded by the gene or fragments thereof.

In another embodiment of the invention, the step of analyzing or determining the level of one or more genes comprises incubation of a sample with a specific antibody. In a more specific aspect of this embodiment, the determination comprises Western blot analysis or immunohistochemistry analysis. According to this embodiment, a sample (e.g., tumor section) is contacted with an antibody having a detectable label. The amount of antibody binding can then be evaluated and compared to a control (e.g., normal cells, a reference value, etc.). In one aspect of this embodiment, the one or more antibodies used to diagnose the presence of colorectal cancer are antibodies that bind to an antigen selected from hCap-E, hCap-C, hCap-D2, hCap-D3, hCap-G, hCap-7G2, hCap-H, hCap-H2, and KIF4A.

In another embodiment of the invention, the step of analyzing or determining the level of one or more genes comprises protein gel electrophoresis.

In another embodiment of the invention, the step of analyzing or determining the level of one or more genes comprises using protein chips (e.g., microarray with antibodies specific for proteins encoded by the one or more genes).

In another embodiment of the invention, the step of analyzing or determining the level of one or more genes comprises ELISA, RIA (radioimmunoassay) or any other enzymatic method.

It is also an object of the present invention to provide a method for the analysis of differential expression in colorectal cancer, wherein the variation in the expression levels of one or more of the described genes is used for predicting the progression of the colorectal cancer or of a premalignant condition thereof, or for predicting the risk of recurrence of the disease. Thus, in one embodiment, the invention provides a method for predicting the progression or recurrence of colorectal cancer, the method comprising: (a) obtaining a sample from a patient, (b) determining the expression level of one or more genes encoding a condensin complex (or condensin complex associated) protein; wherein an increased level of the one or more genes encoding a condensin complex (or condensin complex associated) protein compared to normal tissue or a reference value indicates a higher likelihood of colorectal cancer, cancer progression, and/or recurrence.

It is also an object of the present invention to provide a kit for carrying out the method for the analysis of differential expression, comprising the requisite reagents and additives for determining the variation in the levels of expression of the gene or genes. Thus, in one embodiment, the kit contains reagents for determining, by quantitative PCR, the level of one or more mRNAs corresponding to genes encoding protein that are part of the condensing complex or a protein associated with the condensin complex. In one aspect, the reagents are useful for detecting the expression level of one or more genes chosen from hCap-E, hCap-C, hCap-D2, hCap-D3, hCap-G, hCap-G2, hCap-H, hCap-H2, and KIF4A. In another aspect, the kit contains reagents for analyzing the levels of one or more proteins that are part of and/or associated with the condensing complexes of proteins. For example, the kit can contain antibodies to one or more proteins corresponding to hCap-E, hCap-C, hCap-D2, hCap-D3, hCap-G, hCap-G2, hCap-H, hCap-H2, and KIF4A. The kit can contain markers for normalization or cut-off values, that allow for determining or assessing whether a marker is elevated or not.

It is also an additional object of the present invention to provide a method for the analysis of compounds with therapeutic potential in colorectal cancer, comprising the determination of the capacity of the compounds to decrease the expression levels of one or more of the described genes, with the compounds being compounds tailored according to the sequence information, such as antisense or RNA interference oligonucleotides or others based on the destabilization and elimination of the mRNA or the lack of its translation into protein. Thus, in some embodiments, the invention provides a method for treating colorectal cancer in a patient needing such treatment, comprising administering to the patient an antisense molecule capable of reducing the expression of one or more proteins (or genes) chosen from hCap-E, hCap-C, hCap-D2, hCap-D3, hCap-G, hCap-G2, hCap-H, hCap-H2, and KIF4A. In another embodiment, the invention provides a method for treating colorectal cancer in a patient in need of such treatment, comprising administering to the patient an siRNA/shRNA (or microRNA) molecule capable of reducing the expression of one or more proteins chosen from hCap-E, hCap-C, hCap-D2, hCap-D3, hCap-G, hCap-G2, hCap-H, hCap-H2, and KIF4A. Antisense, siRNA, shRNA and microRNA to these targets are available from commercial sources and readily useable by an ordinary skilled artisan.

In another embodiment, the invention provides a method for identifying compounds that may be useful for treating cancer. According to one aspect of this embodiment, the method involves treating a sample with a test compound and detecting the level of one or more proteins (or mRNA(s) encoding the protein) that are a part of or are associated with the condesin complex. If the test compounds reduces the level of the protein (or mRNA encoding the protein), then it is identified that can be used to treat colorectal cancer.

According to one aspect of this embodiment, the one or more proteins chosen that are part of or are associated with the condensin complex are selected from hCAP-E, hCap-C, hCap-D2, hCap-D3, hCap-G, hCap-G2, hCap-H, hCap-H2, and KIF4A. In one aspect of this embodiment, the level of the protein(s) in the tissue being investigated (e.g., cancerous or suspected of being cancerous) is compared to the level of the protein in normal tissue. If the level of the protein is increased compared to normal tissue, then the tissue is likely to be cancerous or premalignant. In alternative formats, the drug screening assays are designed to examine the effect of the test compound on the biological function of the condensin complex or the individual components of the condensin complex (e.g., DNA repair, cell cycle control, and chromosome condensation).

It is also an additional object of the present invention to provide a method for the analysis of compounds with therapeutic potential in colorectal cancer, comprising the determination of the capacity of the compounds to counteract the variation in the levels of expression of one or more of the described genes, with the compounds being paramagnetic nanoparticles or thermally excited nanoparticles.

It is also an additional object of the present invention to provide a method for the analysis of compounds with therapeutic potential in colorectal cancer, comprising the determination of the capacity of the compounds to counteract the variation in the levels of expression of one or more of the described genes, with the compounds being nanoparticles functionalized with specific antibodies and toxic compounds conveyed in a simple, binary, or modular manner toward the malignant cell.

In some embodiments of the invention, the methods further comprise determining the status of one or more auxiliary genes. The one or more auxiliary genes can be useful for prognostic purposes, predicting response to therapy, choosing therapeutics, diagnosing the disease, predicting the stage of the disease, predicting toxicity to therapies, etc. In one aspect, the auxiliary gene is chosen from tumor suppressors and oncogenes. In one aspect of this embodiment, the one or more tumor suppressors are chosen from p53; the retinoblastoma gene, commonly referred to as Rb1; the adenomatous polyposis of the colon gene (APC); familial breast/ovarian cancer gene I (BRCA1); familial breast/ovarian cancer gene 2 (BRCA2); CDH1 cadherin 1 (epithelial cadherin or E-cadherin) gene; cyclin-dependent kinase inhibitor 1C gene (CDKN1C, also known as p57, KIP2 or BWS); cyclin-dependent kinase inhibitor 2A gene (CDKN2A also known as p16 MTS1 (multiple tumor suppressor 1), TP16 or INK4); familial cylindromatosis gene (CYLD; formerly known as EAC (epithelioma adenoides cysticum)); E1A-binding protein gene (p300); multiple exostosis type 1 gene (EXT1); multiple exostosis type 2 gene (EXT2); homolog of Drosophila mothers against decapentaplegic 4 gene (MADH4; formerly referred to as DPC4 (deleted in pancreatic carcinoma 4) or SMAD4 (SMA- and MAD-related protein 4)); mitogen-activated protein kinase kinase 4 (MAP2K4; also referred to as JNKK1, MEK4, MKK4, or PRKMK4; formerly known as SEK1 or SERK1); multiple endocrine neoplasia type 1 gene (MEN 1); homolog of E. coli MutL gene (MLH1 also known as HNPCC (hereditary non-polyposis colorectal cancer) or HNPCC2; formerly referred to as COCA2 (colorectal cancer 2) and FCC2); homolog of E. coli MutS 2 gene (MSH2 also called HNPCC (hereditary non-polyposis colorectal cancer) or HNPCC1 and formerly known as COCA1 (colorectal cancer 1) and FCC1); neurofibromatosis type 1 gene (NF1); neurofibromatosis type 2 gene (NF2); protein kinase A type 1, alpha, regulatory subunit gene (PRKAR1A, formerly known as PRKAR1 or TSE1 (tissue-specific extinguisher 1)); homolog of Drosophila patched gene (PTCH; also called BCNS); phosphatase and tensin homolog gene (PTEN, also called MMAC1 (mutated in multiple advanced cancers 1), formerly known as BZS (Bannayan-Zonana syndrome) and MHAM1 (multiple hamartoma 1)); succinate dehydrogenase cytochrome B small subunit gene (SDHD; also called SDH4); Swi/Snf5 matrix-associated actin-dependent regulator of chromatin gene (SMARCB1, also referred to as BAF47, HSNFS, SNF5/INI1, SNF5L1, STH1P, and SNR1); serine/threonine kinase 11 gene (STK11 also known as LKB1 and PJS); tuberous sclerosis type 1 gene (TSC1 also known as KIAA023); tuberous sclerosis type 2 gene (TSC2, previously referred to as TSC4); von Hipple-Lindau syndrome gene (VHL); and Wilms tumor 1 gene (WT1, formerly referred to as GUD (genitourinary dysplasia), WAGR (Wilms tumor, aniridia, genitourinary abnormalities, and mental retardation), or WIT-2), DAP-kinase, FHIT, Werner syndrome gene, and Bloom syndrome gene. In another aspect, the one or more tumor suppressors are chosen from, APC, BRCA1, BRCA2, CDH1, CDKN2A, DCC, DPC4 (SMAD4), MADR2/JV18 (SMAD2), MEN1, MLH1, MSH2, MTS1, NF1, NF2, PTCH, p53, PTEN, RB1, TSC1, TSC2, VHL, WRN, TMPRSS2, and WT1. In one aspect of this embodiment, the one or more oncogenes are chosen from K-RAS, H-RAS, N-RAS, EGFR, MDM2, RhoC, AKT1, AKT2, MEK (also called MAPKK), c-myc, n-myc, beta-catenin, PDGF, C-MET, PIK3CA, CDC6, CDK4, cyclin B1, cyclin D1, estrogen receptor gene, progesterone receptor gene, ERG, a member of the ETS family, ET1, ET4, ErbB1, ErbB2 (also called HER2), ErbB3, ErbB4, TGF-alpha, TGF-beta, ras-GAP, Shc, Nck, Src, Yes, Fyn, Wnt, BCL2, and Bmil.

“Determining the status” refers to any method for examining the gene of interest. For example, the gene can be sequenced, the level of expression of the mRNA corresponding to the gene can be determined, the level of expression of the protein can be determined, the methylation of the promoter can be ascertained, the splicing of the gene can be examined, the DNA copy number can be determined, etc.

In some aspects of the invention, the methods further comprise determining the status of one or more genes known to predict response or toxicity to a therapeutic used to treat colorectal cancer. For example, status of DPD (dihydropyrimidine dehydrogenase) can be determined to predict toxic reactions to 5-FU, capcitabine, or related therapeutics. In some aspects of the invention, the UGT 1A1 (Uridine Diphosphate Glucuronosyltransferase) promoter is genotyped to predict toxicity to irinotecan. In some aspects of the invention, the Thymidylate synthase (TS) promoter is genotyped. In some aspects of the invention, the MTFR gene is genotyped. Thus, in one embodiment, the invention provides methods for determining the level of one or more biomarkers corresponding to the condensin complex and determining the status of one or more genes chosen from DPD, UGT1A1, TS, and MTFR.

It is likewise an object of the present invention to provide a pharmaceutical preparation comprising an effective amount of compounds with therapeutic potential identified according to the methods described above and one or more pharmaceutically acceptable excipients.

In addition, it is also an object of the present invention to use compounds with therapeutic potential obtained according to the methods described above for the preparation of a medicinal product for the treatment or prevention of colorectal cancer or of a premalignant condition thereof.

### BRIEF DESCRIPTION OF THE DRAWINGS

FIG. 1A shows hCAP-E localized in interphase cells, represented by the whitish clusters. FIG. 1B is an electron microscopic photograph of a cell nucleus separately showing the heterochromatin and the euchromatin.

FIG. 2 shows the colocalization of hCAP-E in chromosomal regions equivalent to euchromatin (R bands). The same chromosome is shown stained with hCAP-E and stained for G bands. Heterochromatin is identifiable by the darker areas, the one on the right in each of the figures.

FIG. 3 shows the colocalization of hCAP-E with acetylated histone 4 (H4Ac) and the R bands in metaphase chromosomes (light bands in the chromosome).

FIG. 4 shows the results of the Western blot analysis of hCAP-E in normal samples (N) and samples taken from the colorectal cancer tumor (T). Actin was used as loading control.

FIG. 5 relates to an immunohistochemical analysis of colorectal tissue using a specific anti-hCAP-E antibody. The picture shows the area corresponding to the normal crypt (N) and the area corresponding to the colon adenocarcinoma (T), which exhibits greater expression of hCAP-E.

FIG. 6 relates to an immunohistochemical analysis in normal colon crypts using a specific anti-hCAP-E antibody, in which it is observed that the pluripotent stem cells exhibit a stronger specific staining for hCAP-E than for goblet cells.

FIG. 7 shows the results of the real-time PCR analysis of other proteins in the condensin complex and the associated protein KIF4A. The sample analyzed was sample 67T, which had previously been observed, when compared to its corresponding normal sample, to over-express hCAP-E. The analysis was carried out in triplicate and ribosomal 18S was used as internal control.

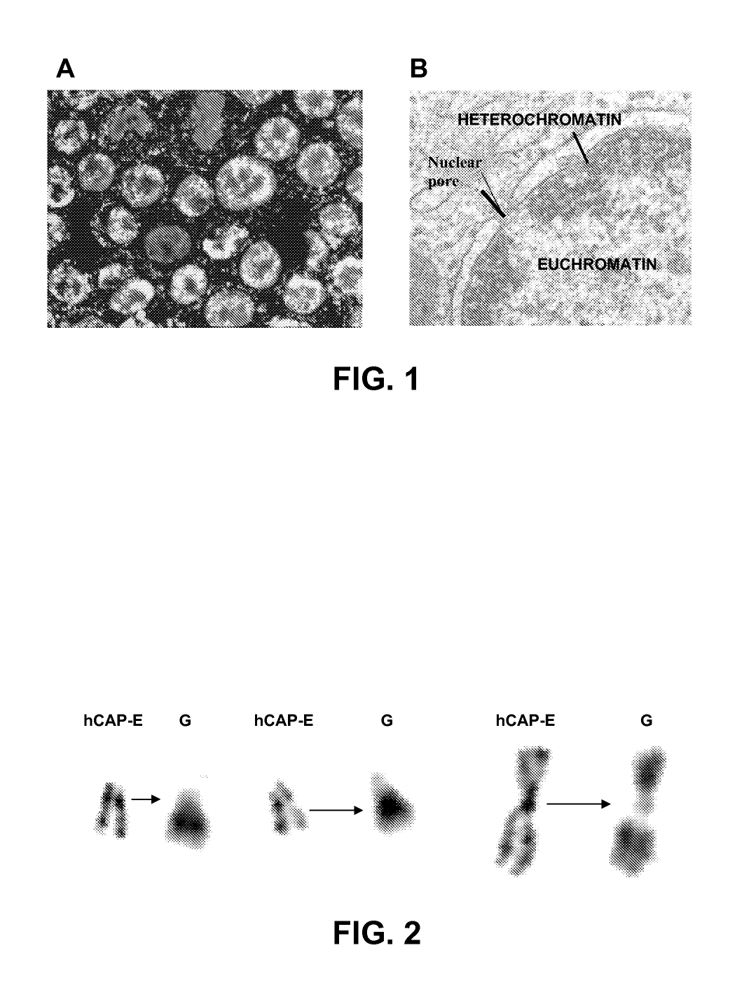

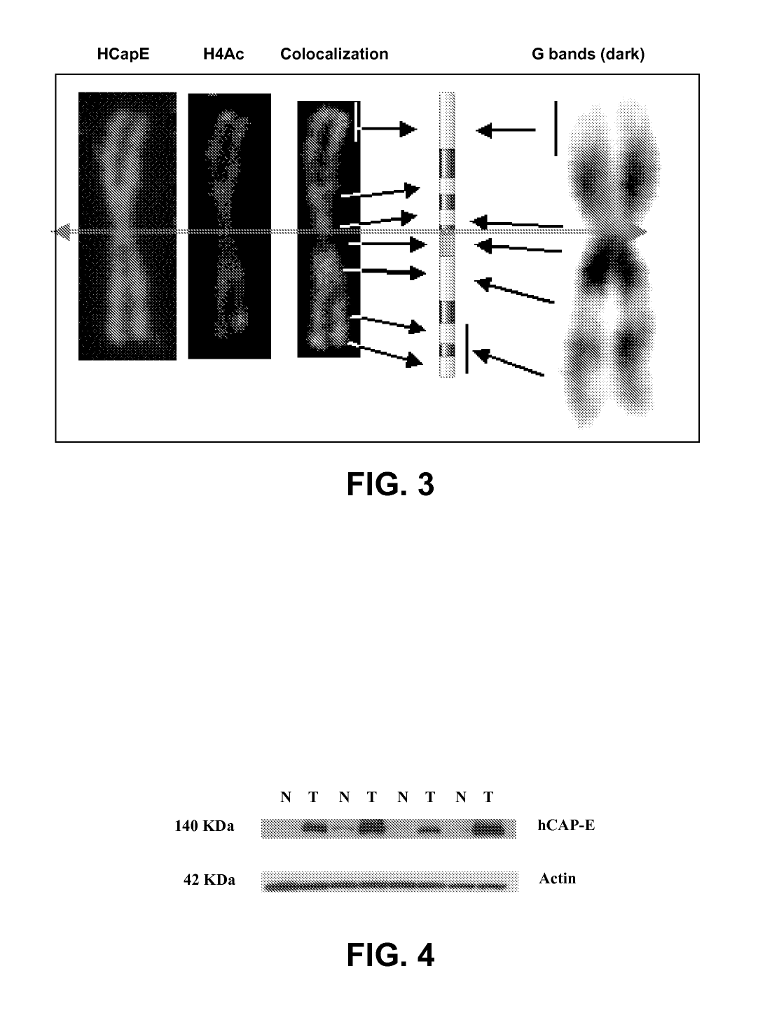

### DETAILED DESCRIPTION OF THE INVENTION

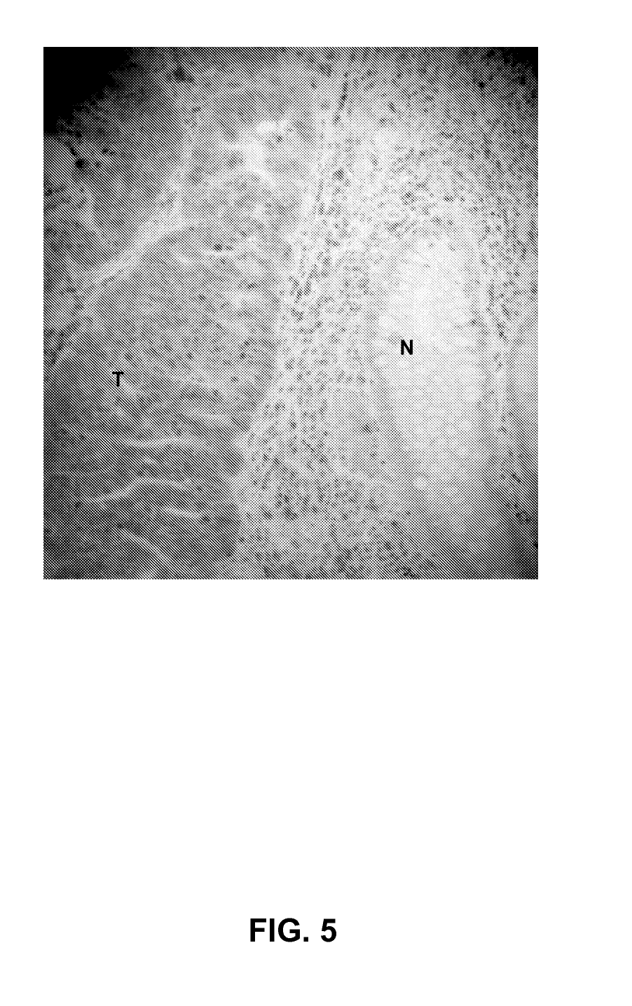

The present invention is based on the over-expression of proteins of the condensin complex and associated proteins observed in colorectal cancer patients. The data showed an over-expression of hCAP-E in the Western blot analysis using specific anti-hCAP-E antibodies in colorectal cancer samples in comparison with samples of normal tissue, regardless of its tumor stage, with a 90% incidence of tumor over-expression (18/20) (FIG. 4). This over-expression was also observed in all immunohistochemical analyses of colorectal cancer tumor tissue (FIG. 5).

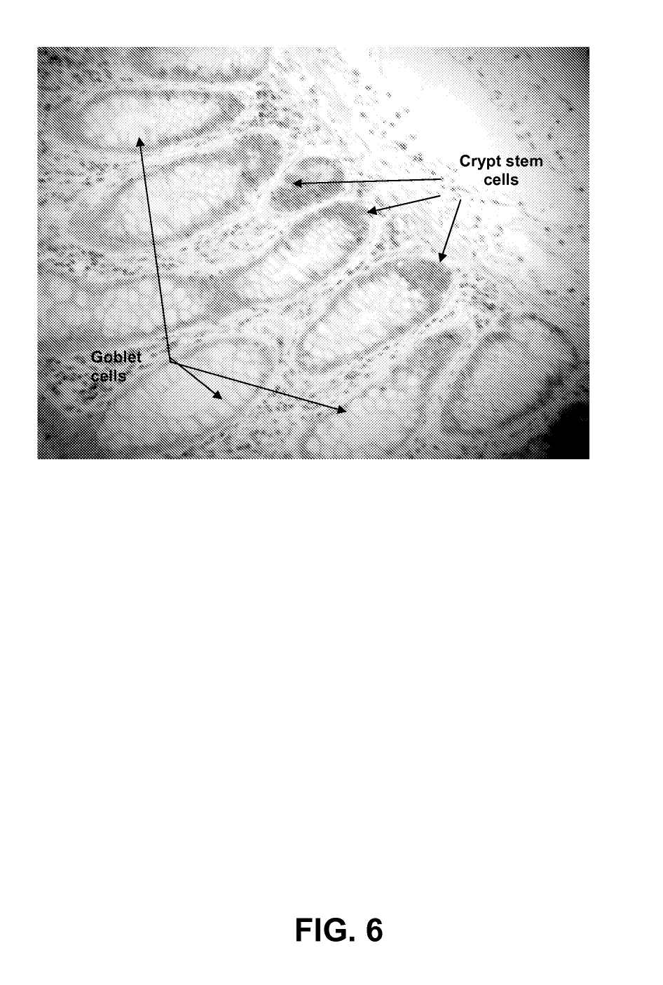

Likewise it was observed that the expression of hCAP-E is also specific for pluripotent (stem) cells of the colon crypt (FIG. 6), which are the undifferentiated cells from which colorectal tumors originate. Their expression pattern indicates that the latter virtually disappears as the pluripotent cells are transformed into epithelial cells (goblet cells) in a normal crypt to form the epithelium, so that it could be regarded as a marker of cell differentiation.

Table 3 shows the levels of over-expression of hCAP-E in colorectal cancer according to the staging.

TABLE 3
Over-expression of hCAP-E in colorectal
cancer according to the staging

| CASE | STAGE | hCAP-E |
|---|---|---|
| 94T | I | + |
| 78T | II | + |
| 100T | II | + |
| 67T | II | + |
| 85T | II | + |
| 60T | III | + |
| 162T | III | + |
| 141T | III | + |
| 38T | III | + |
| 55T | III | + |
| 66T | III | − |
| 88T | III | + |
| 91T | III | + |
| 213T | IV | = |
| 129T | IV | + |
| 35T | IV | + |
| 31T | IV | + |
| 36T | IV | + |
| 86T | IV | + |
| 137T | IV | + |

The incidence of over-expression (+) was 90% (18 out of 20 tumors). Only one case was observed in which expression was below normal (−) and one case in which normal tissue and tumor tissue showed similar expression levels (=).

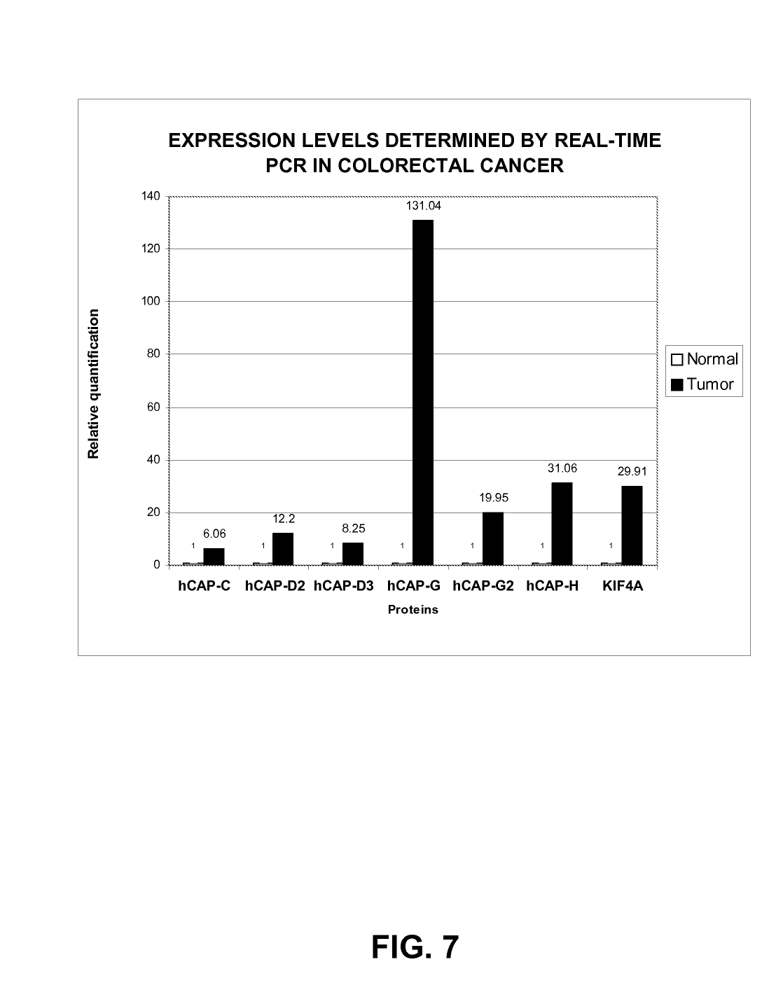

The expression levels of other proteins forming the condensin complex, such as hCap-C, hCap-D2, hCap-D3, hCap-G, hCap-G2 and hCap-H, were likewise analyzed by real-time PCR, as were other associated proteins that interact with the complex, such as KIF4A, in certain tumors in which over-expression of hCap-E had been observed. The results indicated that all the analyzed proteins showed increased expression levels in tumor tissue in comparison with the levels in normal tissue (FIG. 7). It is, therefore, concluded that all the proteins that make up the condensin complex and other proteins interacting with the complex are over-expressed in tumors.

Accordingly, the proteins forming the condensin complex and other associated proteins that interact with it may be used as markers of colorectal cancer or of a premalignant condition thereof, potentially acting as a diagnostic marker and/or a marker for recommending colonoscopy.

These proteins can also act both as markers of pluripotent stem cells of the colon crypt and as markers of cell differentiation. Moreover, these proteins can be histological markers of cancer and/or be useful in imaging analysis systems.

In addition, these proteins can constitute direct or indirect therapeutic targets, enabling tumor-targeted anticancer treatments to hit the tumor through interactions with any of these proteins or by modulation of their expression levels.

Interestingly, it has recently been reported that the expression levels of the proteins in the condensin complex do not vary throughout the cell cycle and, therefore, do not vary during mitosis (Takemoto et al., 2004). So, the fact that high levels of these proteins are found in cancer cells, as described in the present invention, cannot be attributed simply to an increase in replication activity (mitosis) of the tumor cell, but rather to actual relative over-expression due to the development of the disease.

Therefore, the present invention shows that there is a complete association between the expression levels of the proteins that make up the condensin complex and other proteins associated with the complex and the presence of colorectal cancer, whatever its stage of development, which means that there is now a new molecular tool available that enables the disease to be diagnosed even in its earliest stages, something that is not possible using methods currently available.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., T. Maniatis et al. (1982), Molecular Cloning: A Laboratory Manual (Cold Spring Harbor Laboratory, Cold Spring Harbor, N.Y.); J. Sambrook et al. (1989), Molecular Cloning: A Laboratory Manual, 2nd Ed. (Cold Spring Harbor Laboratory, Cold Spring Harbor, N.Y.); F. M. Ausubel et al. (1992), Current Protocols in Molecular Biology (J. Wiley and Sons, NY); D. Glover (1985), DNA Cloning, I and II (Oxford Press); R. Anand (1992), Techniques for the Analysis of Complex Genomes (Academic Press); G. Guthrie and G. R. Fink (1991), Guide to Yeast Genetics and Molecular Biology (Academic Press); Harlow and Lane (1988), Antibodies: A Laboratory Manual (Cold Spring Harbor Laboratory, Cold Spring Harbor, N.Y.; W. B. Jakoby and I. H. Pastan (eds.) (1979), Cell Culture: Methods in Enzymology, Vol. 58 (Academic Press, Inc., Harcourt Brace Jovanovich (NY)); Nucleic Acid Hybridization (B. D. Hames and S. J. Higgins eds. 1984); Transcription and Translation (B. D. Hames and S. J. Higgins eds. 1984); Culture of Animal Cells (R. I. Freshney, Alan R. Liss, Inc., 1987); Immobilized Cells and Enzymes (IRL Press, 1986); B. Perbal (1984), A Practical Guide To Molecular Cloning; the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell and Molecular Biology (Mayer and Walker, eds., Academic Press, London, 1987).

### EXAMPLES

Below is described a preferred, though not exclusive, embodiment of the invention.

### Patient Samples

Biopsies of normal and cancerous tissue were obtained from 20 patients diagnosed with colorectal cancer. The surgically obtained samples were immediately frozen in liquid nitrogen and kept at −80° C. for later extraction of proteins and RNA. In addition, histological sections were prepared for immunohistochemical testing. The clinicopathological characteristics were recorded, including the stage and differentiation grade of the tumors, as well as at least a three-year follow-up to detect any early recurrence.

Analysis of the Expression Levels of hCAP-E

Western blot: The proteins were extracted from the samples of normal and cancerous colorectal tissue by standard methods, using RIPA lysis buffer. Ninety μg of protein were fractionated in 10% SDS-PAGE gel, transferred to a nitrocellulose membrane (BioRAD, USA), blocked with 5% milk in TBS-T, and hybridized with a primary anti-hCAP-E antibody (Abcam, UK) diluted 1:2500 in blocking solution, and then with a secondary anti-rabbit antibody (Dako Cytomation, Denmark) in a 1:500 dilution. The chemiluminescent signal was detected using the ECL kit (Amersham, USA) and the expression levels of the tumor samples were compared with their normal counterparts. Finally, the membrane was rehybridized with anti-actin antibody (Invitrogen, USA), which was used as load control. As can be seen from FIG. 4, all the tumor samples exhibited increased hCAP-E expression levels.

Immunohistochemistry: The histological sections taken from the tissues of patients with colorectal cancer were deparaffinized with xylene and rinsed in decreasing series of ethanol and distilled water. The sections were treated with citrate buffer at pH 6 (five minutes at 800 W and ten minutes at 450 W in a microwave) the endogenous peroxidase was blocked with H₂O₂, and they were then hybridized with the primary anti-hCAP-E antibody (Abcam, UK). For the immunohistochemical analysis, the EnVision+Dual Link System kit (Dako Cytomation, Denmark) was used in accordance with the manufacturer's recommendations. Finally, the hCAP-E expression levels in areas of normal and cancerous tissue were compared. FIG. 5 shows that hCAP-E expression is greater in cancerous than in normal tissue.

Analysis of the Expression Levels of other Proteins of the Condensin Complex and Associated Proteins

Real-time PCR: The mRNA levels corresponding to other proteins of the condensin complex and associated proteins were quantified by real-time PCR, for which purpose RNA was extracted from samples of normal and cancerous colorectal tissue kept at −80° C., using Trizol (Invitrogen, USA). Ten jig of RNA was retro-transcribed using the High Capacity cDNA Archive kit (Applied Biosystems, USA) and amplified with TaqMan® Gene Expression Assays (Applied Biosystems, USA) for hCAP-C, hCAP-D2, hCAP-D3, hCAP-G, hCAP-G2, hCAP-H, and KIF4A, respectively. The amplification reaction was carried out using TaqMan Universal PCR Master Mix in the 7500 Real-Time PCR System (both from Applied Biosystems, USA). The relative mRNA levels of each gene were quantified using the ΔΔC_{T} method and the program associated with the system. The test was carried out in triplicate and 18S rRNA was used as the endogenous control, and the expression of normal tissue and of cancerous tissue from the same patient was compared. As FIG. 7 shows, for all the genes analyzed, the cancerous samples exhibited substantially elevated mRNA levels when compared to the control samples.

### REFERENCES

Hagstrom K. and B. J. Meyer. Condensin and cohesin: more than chromosome compactor and glue. Nat. Rev. Genet. 2003 July 4:520-534.
Hirano T. The ABC of SMC proteins: two-armed ATPases for chromosome condensation, cohesion and repair. Genes & Development, 2002 16:399-341.
Legagneux V., F. Cubizolles, and E. Watrin. Multiple roles of condensins: a complex story. Biology of the Cell, 2004 96:201-213.
Hirano T. and T. J. Mitchison. A heterodimeric coiled-coil protein required for mitotic chromosome condensation in vitro. Cell 1994 Nov. 4; 79(3):449-58.
Ono T., Y. Fang, D. L. Spector, and T. Hirano. Spatial and temporal regulation of Condensins I and II in mitotic chromosome assembly in human cells. Mol. Biol. Cell. 2004 July; 15(7):3296-308.
Ono T., A. Losada, M. Hirano, M. P. Myers, A. F. Neuwald, and T. Hirano. Differential contributions of condensin I and condensin II to mitotic chromosome architecture in vertebrate cells. Cell. 2003 Oct. 3; 115(1):109-21.
Takemoto A., K. Kimura, S. Yokoyama, and F. Hanaoka. Cell cycle-dependent phosphorylation, nuclear localization, and activation of human condensin. J. Biol. Chem. 2004 Feb. 6; 279(6):4551-9.

What is claimed is:

1. A method for diagnosing colorectal cancer, said method comprising determining the level of one or more biomarkers in a sample, wherein said one or more biomarkers corresponds to a member of the condensin complexes or a protein associated with the protein complex, wherein an alteration in the level of the one or more biomarkers as compared to control indicates colorectal cancer or pre-malignant colorectal cancer state.

2. The method of claim 1, wherein said one or more biomarkers are chosen from a nucleic acid, a DNA, a RNA, and a protein.

3. The method of claim 1, wherein said one or more biomarkers are selected from the group consisting of hCap-E, hCap-C, hCap-D2, hCap-D3, hCap-G, hCap-G2, hCap-H, hCap-H2, and KIF4A.

4. The method of claim 1, wherein the alteration in the level of one or more biomarkers results in an increase in the level of the mRNA.

5. The method of claim 1, wherein the alteration in the level of one or more biomarkers results in an increase in the level of the protein.

6. The method of claim 1, wherein the sample is isolated from cells obtained by biopsy or any other method of extraction.

7. The method of claim 1, wherein the determining the level comprises analyzing the sample for the level of DNA or RNA.

8. The method of claim 1, wherein said determining the level is carried out by (1) PCR amplification, SDA amplification, or any other method of nucleic acid amplification, (2) using a nucleic acid microarray, (3) gel electrophoresis, (4) transfer to a membrane and hybridization with a specific probe, and (5) diagnostic imaging.

9. The method of claim 1, wherein the determining the level comprises analyzing the sample for the level of the protein.

10. The method of claim 1, wherein the analysis is carried out by (1) incubation with a specific antibody, (2) Western blot, (3) immunohistochemistry, (4) gel electrophoresis, (5) microarray, (6) ELISA, and (7) diagnostic imaging.

11. The method of claim 1, wherein the variation in the expression levels of the gene or genes is used to predict the progression of the colorectal cancer or of a premalignant condition thereof, for predicting the risk of recurrence, and/or determining the type of therapy.

12. A kit for the diagnosis, prognosis, and/or prediction of recurrence of colorectal cancer, said kit comprising reagents and additives for determining the variation in the one or more biomarkers corresponding to proteins that are part of condensin complex or are associated with the condensin complex.

13. The kit of claim 12, wherein said one or more biomarkers corresponding to the condensin complex or are associated with the condensin complex are chosen from hCap-E, hCap-C, hCap-D2, hCap-D3, hCap-G, hCap-G2, hCap-H, hCap-H2, and KIF4A.

14. The method of claim 1, wherein said biomarker corresponds to hCap-E nucleic acid or protein.

15. The method of claim 1, wherein said biomarker corresponds to hCap-C nucleic acid or protein.

16. The method of claim 1, wherein said biomarker corresponds to hCap-D2 nucleic acid or protein.

17. The method of claim 1, wherein said biomarker corresponds to hCap-D3 nucleic acid or protein.

18. The method of claim 1, wherein said biomarker corresponds to hCap-G nucleic acid or protein.

19. The method of claim 1, wherein said biomarker corresponds to hCap-G2 nucleic acid or protein.

20. The method of claim 1, wherein said biomarker corresponds to hCap-H nucleic acid or protein.

21. The method of claim 1, wherein said biomarker corresponds to hCap-H2 nucleic acid or protein.

22. The method of claim 1, wherein said biomarker corresponds to KIF4A nucleic acid or protein.

23. A method for screening for compounds with anti-colorectal cancer activity, said method comprising contacting a cell with a test compound and determining if said compound either reduces the level of expression of one or more genes that encode a component of the condensin complex or a protein associated with the condensin complex or alters the biological function of the condensin complex.

24. The method of claim 23, wherein the biological function of the condensin complex is the ability to mediate repair of DNA damage.

25. The method of claim 23, wherein the biological function of the condensin complex is the ability to mediate cell cycle control.
